(19) United States
(12) Patent Application Publication
(10) Pub. No.: US 2005/0019438 A1
(43) Pub. Date: Jan. 27, 2005

(54) Hop extracts and use thereof in the production of a medicament having estrogenic properties

(76) Inventors: Cedric Bourges-Sevenier, Les Ponts De Ce (FR); Olivier Fahy, Gardonne (FR)

Correspondence Address: YOUNG & THOMPSON, 745 SOUTH 23RD STREET, 2ND FLOOR, ARLINGTON, VA, 22202, US

(21) Appl. No.: 10/475,662

(22) PCT Filed: Apr. 15, 2002
(86) PCT No.: PCT/FR02/01295

(30) Foreign Application Priority Data
Apr. 23, 2001 (FR) 01/05463

(51) Int. Cl. A61K 35/78; A61K 31/353; A61K 31/045

(52) U.S. Cl. 424/778; 514/456; 514/729

(57) ABSTRACT

A hop extract obtained from the female hop cones of certain varieties of hops, primarily includes the following prenylflavanoid constituents: xanthohumol, isoxanthohumol and 8-prenylnaringenine, in defined weight proportions. The extract is used in the production of a medicament having estrogenic properties, used to treat physiological disorders related to perimonopause or menopause such as hot flashes. The medicament can also be used in dietary compositions of food supplements and cosmetic compositions.

The present invention relates to hop extracts and to their use in the production of a medicament having estrogenic properties.

Female hop cone inflorescences have been used for a long time in the production of beer. They are rich in mineral substances (especially potassium salts) and also contain tannins, amines, pectins, traces of histamine, and many polyphenols, including flavonoids: rutoside, quercitroside, astragaloside, and also chalcones and isoprenylated flavanones, such as xanthohumol, isoxanthohumol or 8-prenylnaringenin.

The estrogenic activity of hops has already been recognized and attested to through use in conventional medicine. Recently, it has been possible to identify the beneficial properties of xanthohumol against osteoporosis (U.S. Pat. No. 5,679,716) and also those of 8-isopentenylnaringenin (Planta Medica 1998, 64, p. 516-519). However, polyphenolic extracts of various varieties of hops studied by De Keukeliere et al., in Pharm. Pharmacol. Lett. 1997, 2/3, p. 83-86, have shown estrogenic activity without it being possible for this activity to be attributed to xanthohumol or to desmethylxanthohumol.

Surprisingly, the inventors have discovered that, if a hop extract is produced from female inflorescences derived from certain hop varieties, and in particular if it contains, among the prenylflavonoids, mainly the following three constituents: xanthohumol, isoxanthohumol and 8-prenylnaringenin (or 8-isopentenylnaringenin), it exhibits an estrogenic activity greater than conventional hop extracts.

The present invention therefore relates to a hop extract obtained from female Humulus lupulus hop cones derived from at least one of the following hop varieties: brewer's gold, cascade, cluster, colombus, galena, northern brewer, nugget, zeus, magnuim, perle or taurus, and containing, among the prenylflavonoids, mainly the following three constituents: xanthohumol, isoxanthohumol and 8-prenylnaringenin.

Preferably, the extract also contains at least one other prenylflavonoid from: desmethylxanthohumol, tetrahydroxygeranylchalcone, 5-prenylxanthohumol, dehydrocycloxanthohumol, dehydrocycloxanthohumol hydrate, 6-prenylnaringenin, 8-geranylnaringenin, 6-geranyl-naringenin and 3′-geranylchalconaringenin.

Advantageously, the extract according to the invention contains at least 3% by weight of prenylflavonoids.

The estrogenic properties are particularly demonstrated when the weight proportions of said three constituents in 100 g of dry extract are as follows: from 1 to 30 g of xanthohumol, from 0.01 to 50 g of isoxanthohumol and from 0.5×10⁻³ to 10 g of 8-prenylnaringenin, and preferably within the ranges of 3 to 15 g of xanthohumol, of 3 to 30 g of isoxanthohumol and of 0.01 to 5 g of 8-prenylnaringenin.

The extract according to the present invention can be used for producing a medicament having estrogenic properties, and in particular for the treatment of hormone variations related to perimenopause or menopause, which cause physiological modifications leading to problems such as hot flushes, problems of mood and with memory, urinary incontinence, loss of integrity of the structure of the tissue supporting the skin, hair loss, a decrease in sweat gland activity, vaginal dryness, osteoporosis, cardiovascular diseases, etc.

Such a medicament can be intended in particular for the treatment of hot flushes occurring during perimenopause or menopause. The dosages are, for example, of the order of 3 mg/kg bodyweight and per day.

The specific mixture of the three xanthohumol, isoxanthohumol and 8-prenylnaringenin molecules in the following relative proportions: from 1 to 30 g of xanthohumol, from 0.01 to 50 g of isoxanthohumol and from 0.5×10⁻³ to 10 g of 8-prenylnaringenin, and preferably within the ranges of 3 to 15 g of xanthohumol, of 3 to 30 g of isoxanthohumol and of 0.01 to 5 g of 8-prenylnaringenin, per 100 g of dry extract, can also be used to produce a medicament having estrogenic properties or activities, and intended in particular for the treatment of physiological disorders related to perimenopause and menopause, such as hot flushes.

The abovementioned mixture, or the extract according to the present invention, can also be used in dietetic compositions (for example in the form of powders, gelatin capsules, tablets, capsules, vials, drinks), in food supplements or in cosmetic compositions (for example in the form of creams, gels, lotions, etc.).

The present invention will be illustrated by the following examples:

### EXAMPLE 1

a) Extraction

Female Humulus lupulus hop cones derived from the variety northern brewer (origin hallertauer) are cut up into pieces of a few centimeters. A solid/liquid extraction of these cones is performed with an organic solvent (or a mixture of solvents). The solvent is then filtered in order to remove the thoroughly extracted plant material. The filtrate is recovered and is subjected to a liquid/liquid extraction, and then the second phase is recovered and subjected to physical separation processes (of the membrane type) in order to remove mainly the macromolecules present. The liquid obtained is finally dried, finely ground, and packaged.

b) In Vitro Assay

The in vitro assay used was developed by Littlefield in 1990 (Endocrinology 1990, 127, p. 2757-2762).

The variant of the cell line used is sensitive to estrogens, to which it responds not through a proliferative activity, but through an activity which stimulates an enzyme, alkaline phosphatase. This assay provides, in a sensitive and specific manner, information on the estrogenic activity since, among steroids, only the estrogens respond to this assay and succeed in stimulating the enzyme activity. The estrogenic activity can also be triggered by non-steroidal substances having a “estrogen-like” effect, such as phytoestrogens. The estrogenic responses obtained below are blocked by the reference anti-estrogen brought into contact, which indicates an estrogen receptor-mediated activity.

The model is an Ishikawa cell line (Var. I) of human endometrial adenocarcinoma (established by Nishida: Acta Obstet Gynaec Jap 1985, 37: 1103-1111).

The assay is based on the identification of alkaline phosphatase (AP) activity demonstrating estrogenic activity.

Specifically, alkaline phosphatase hydrolyzes p-nitrophenol phosphate to p-nitrophenol, which gives a colored reaction.

The assay consists in measuring the absorbance at 405 nm after incubation for 72 h.

Two controls were used:
positive control=17β-estradiol,
negative control=antiestrogen ICI182, 780 from Zeneca.

The extracts brought into contact at a concentration of 2 micrograms/ml are:
soybean extract containing 7% by weight of isoflavones
soybean extract containing 20% by weight of isoflavones
“conventional” hop extract
hop extract according to the invention containing the following respective proportions of xanthohumol, isoxanthohumol and 8-prenylnaringenin: 5, 7 and 0.06% by weight.
   c) Results

They are given in table I below:

TABLE 1

| EXTRACTS | ABSORBANCE at 405 nm |
|---|---|
| Soybean extract containing | 0.300 |
| 7% of isoflavones | |
| Soybean extract containing | 0.405 |
| 20% of isoflavones | |
| Hop extract according to | 0.360 |
| the invention | |
| “Conventional” hop extract | 0.210 |

17β-Estradiol gives a curve representing the expected dose-effect relationship (estrogenic by activation of alkaline phosphatase).

There is also, moreover, a dose-effect relationship when soybean extracts containing an increasing content of isoflavones are used. This result validates the recognized estrogenic activity of isoflavones, but especially validates the assay set up for non-steroidal compounds with phytoestrogenic activity.

As regards the extracts, the observations are as follows:
The soybean extract containing 7% of isoflavones gives a weak but significant estrogenic response.
The soybean extract containing 20% of isoflavones gives a considerable estrogenic response which is significantly greater than that found for the soybean extract containing 7% of isoflavones, validating the recognized isoflavone dose-related estrogenic effect of soybean and validating the activity of the assay.
The “conventional” hop extract is a hop extract commonly found commercially, which shows little activity, which is furthermore weaker than that observed with the soybean extract containing 7% of soybean isoflavones. It is therefore only of moderate value with regard to its phytoestrogenic potential.
The hop extract according to the invention has an estrogenic activity which is much greater than the conventional commercial hop extract and than a soybean extract containing 7% of isoflavones.
The hop extract according to the invention gives phytoestrogenic results close to those obtained with a soybean isoflavone extract containing 20% of isoflavones.
   d) Conclusion

It may be deduced from this example that the hop extract according to the invention gives estrogenic activities similar to those obtained with a soybean extract containing 20% by weight of isoflavones, and greater than those of a conventional hop extract.

### EXAMPLE 2

Humulus lupulus hop extracts derived from various varieties were prepared and assayed according to a protocol identical to that of example 1. Their proportions of xanthohumol, isoxanthohumol and 8-prenylnaringenin were assayed by HPLC.

The results are given in table II below:

TABLE II

| | | % in dry extract | | | Absorbance |
|---|---|---|---|---|---|
| Variety | Origin | X | IX | 8PN | at 405 nm |
| Hallertau | Hallertauer | 4 | 8 | 0.01 | 0.020 |
| Hersbruck | Hallertauer | 2.8 | 9 | 0.03 | 0.023 |
| Saaz | Saazer | 3 | 4 | 0.003 | 0.090 |
| Horizon | Osu | 5.5 | 2 | 0.1 | 0.100 |
| Cluster | Idaho | 4 | 2 | 0.003 | 0.160 |
| Colombus | Washington | 6 | 7 | 0.02 | 0.200 |
| Cascade | Washington | 2 | 15 | 0.005 | 0.210 |
| Galena | Idaho | 5 | 0.02 | 0.003 | 0.270 |
| Perle | Oregon | 2 | 12 | 0.0007 | 0.280 |
| Taurus | Washington | 8 | 20 | 0.015 | 0.300 |
| Brewer's | Hallertauer | 8 | 0.5 | 0.002 | 0.300 |
| gold | | | | | |
| Nugget | Oregon | 7 | 0.1 | 0.08 | 0.320 |
| Northern | Hallertauer | 5 | 7 | 0.06 | 0.360 |

X = xanthohumol
IX = isoxanthohumol
8PN = 8-prenylnaringenin

These results show that most of the varieties tested exhibit an absorbance equal to or greater than that of the conventional hop extract (0.210), i.e. equivalent or superior estrogenic properties.

It is noted that the estrogenic properties are particularly high when the inflorescences extracted are derived from at least one of the following hop varieties: galena, perle, taurus, brewer's gold, nugget and northern brewer. Similar results were also obtained with the varieties Zeus and Magnuim.

### EXAMPLE 3

A size-1 gelatin capsule containing the following components:
60 mg of extract according to the invention (var. northern brewer)
200 mg of maltodextrin
10 mg of colloidal silica
5 mg of magnesium stearate
   was ingested, twice a day, by a patient weighing 60 kg. After treatment for one month, this patient noted a clear decrease in hot flushes.

1. A hop extract obtained from female Humulus lupulus hop cones derived from at least one of the following hop varieties: brewer's gold, cascade, cluster, colombus, galena, northern brewer, nugget, zeus, magnuim, perle or taurus, and containing, among the prenylflavonoids, mainly the following three constituents: xanthohumol, isoxanthohumol and 8-prenylnaringenin.

2. The hop extract as claimed in claim 1, characterized in that it contains at least one other prenylflavonoid from: desmethylxanthohumol, tetrahydroxygeranylchalcone, 5-prenylxanthohumol, dehydrocycloxanthohumol, dehydrocycloxanthohumol hydrate, 6-prenylnaringenin, 8-geranylnaringenin, 6-geranylnaringenin and 3′-geranylchalconaringenin.

3. The hop extract as claimed in claim 1, characterized in that it contains at least 3% by weight of prenylflavonoids.

4. The extract as claimed in claim 1, characterized in that it contains said three constituents in the following weight proportions: from 1 to 30 g of xanthohumol, from 0.01 to 50 g of isoxanthohumol and from 0.5×10⁻³ to 10 g of 8-prenylnaringenin, in 100 g of dry extract.

5. The extract as claimed in claim 1, characterized in that it contains said three constituents in the following weight proportions: from 3 to 15 g of xanthohumol, from 3 to 30 g of isoxanthohumol and from 0.01 to 5 g of 8-prenylnaringenin, in 100 g of dry extract.

6. The use of the extract as claimed in claim 1, for producing a medicament having estrogenic properties.

7. The use of the extract as claimed in claim 1, for producing a medicament intended for the treatment of physiological disorders related to perimenopause and menopause.

8. The use as claimed in claim 7, for producing a medicament intended for the treatment of hot flushes.

9. A method for producing a medicament having estrogenic properties comprising the steps of mixing xanthohumol, isoxanthohumol and 8-prenylnaringenin in the following relative weight proportions in 100 g of dry extract: from 1 to 30 g of xanthohumol, from 0.01 to 50 g of isoxanthohumol and from 0.5×10⁻³ to 10 g of 8-prenylnaringenin.

10. A method for producing a medicament for the treatment of physiological disorders related to perimenopause and menopause comprising the steps of mixing xanthohumol, isoxanthohumol and 8-prenylnaringenin in the following relative weight proportions in 100 g of dry extract: from 3 to 15 g of xanthohumol, from 3 to 30 g of isoxanthohumol and from 0.01 to 5 g of 8-prenylnaringenin.

11. The method as claimed in claim 9, for producing a medicament for the treatment of hot flashes.

12. A dietetic composition containing a mixture of xanthohumol, isoxanthohumol and 8-prenylnaringenin in the following relative weight proportions in 100 g of dry extract: from 1 to 30 g of xanthohumol, from 0.01 to 50 g of isoxanthohumol and from 0.5 to 10 g of 8-prenylnaringenin.

13. A food supplement containing a mixture of xanthohumol, isoxanthohumol and 8-prenylnaringenin in the following relative weight proportions in 100 g of dry extract: from 1 to 30 g of xanthohumol, from 0.01 to 50 g of isoxanthohumol and from 0.5 to 10 g of 8-prenylnaringenin.

14. A cosmetic composition containing a mixture of xanthohumol, isoxanthohumol and 8-prenylnaringenin in the following relative weight proportions in 100 g of dry extract: from 1 to 30 g of xanthohumol, from 0.01 to 50 g of isoxanthohumol and from 0.5 to 10 g of 8-prenylnaringenin.
